(19) United States
(12) Patent Application Publication
(10) Pub. No.: US 2010/0069819 A1
(43) Pub. Date: Mar. 18, 2010

(54) Minimally invasive gastrointestinal bypass

(76) Inventor: Michael D. Laufer, Menlo Park, CA (US)

Correspondence Address: Nixon Peabody LLP, P.O. Box 60610, Palo Alto, CA, 94306, US

(21) Appl. No.: 11/546,458

(22) Filed: Oct. 10, 2006

Related U.S. Application Data
(63) Continuation of application No. 10/687,954, filed on Oct. 17, 2003, now abandoned.

(51) Int. Cl. A61F 2/04

(52) U.S. Cl. 604/8

(57) ABSTRACT

A solution is provided for modifying the location at which bodily fluids interact with nutrients in a gastrointestinal tract having a conduit with a first end and a second end. The first end is configured to divert bodily fluids from an entrance within a gastrointestinal tract to a location downstream from the entrance. The solution also provides for a means for attaching the second end to the entrance.

### FIELD OF THE INVENTION

The present invention relates to medical surgeries. More particularly, the present invention relates to medical surgeries on the digestive system.

### BACKGROUND OF THE INVENTION

Twenty million Americans are markedly overweight, and only about seven million are currently eligible for surgery to reconstruct their gastrointestinal (GI) tract to make it possible for them to lose weight. These procedures are reserved for the severely obese because they have a number of significant complications, including the risk of death. In these patients, it is estimated that their annual mortality is as high as 30%-50%, which justifies the use of these risky procedures. No procedure exists for the less obese people that would like to lose between 20 to 50 pounds of weight.

FIG. 1 is an illustration of the digestive system. The digestive tract is a disassembly line in which food becomes less and less complex and its nutrients become available to the body. Food 10 enters the mouth 12 and is chewed and mixed with saliva with the tongue. The food 10 is then swallowed and enters the pharynx 14 where propulsion causes the food to continue through the digestive tract to the esophagus 16. As the food continues through the digestive tract, it is mixed with other fluids to create a fluid of food. Below the esophagus 16, the (GI) tract expands to form the stomach 18. The stomach 18 is where the mechanical and chemical breakdown of proteins occurs such that when the food leaves the stomach, it is converted into a substance called chyme. From the stomach 18, the food fluid or chyme, enters the small intestine 20 where digestion is completed with the aid of secretions from the liver 22 and the pancreas 24.

Bile is made in the liver and stored in the gall bladder 26. Bile is a complex mixture of emulsifiers and surfactant that are needed in the body to absorb fat. Without bile, dietary fat is relatively insoluble and passes out in the feces. Pancreatic enzymes are made in the pancreas 24 and are necessary to digest and absorb proteins, and to a lesser degree, carbohydrates. The pancreatic enzymes move from the pancreas to the intestine through the pancreatic duct 28, which in most individuals combines with the bile duct 32 from the gall bladder 26 to form a common duct that enters the intestine through the Ampula of Vater 30. However, in some individuals, the bile duct 32 and pancreatic duct 28 remain separate and enter the small intestine 20 separately.

As the food fluid journeys through the small intestine 20, digested foodstuff, such as fats, are absorbed through the mucosal cells into both the capillary blood and the lacteal 38. Other digested foodstuffs, such as amino acids, simple sugars, water, and ions are absorbed by the hepatic portal vein 40. From the small intestine 20, the remainder of the food fluid enters the large intestine 42 whose major function is to dry out indigestible food residues and eliminate them from the body as feces 44 through the anal canal 46.

Current gastrointestinal tract surgeries require incisions to be made into the abdomen in order to attach the distal small intestine to the stomach and to make the stomach smaller. This procedure is sometimes called “Roux-en-Y” or gastro jejunal bypass with gastric reduction. The procedure is commonly performed through a large midline abdominal incision, although some surgeons have developed adequate skill to perform the procedure through a number of smaller incisions in a laparoscopic trimmer with cameras and instruments inserted through the holes for visualization. Both methods cause weight loss through bypass by reducing the effective length of intestine available for the absorption of food and the stomach is reduced in size so that the patient cannot eat a lot of food. However, both methods require anesthesia (usually general), a prolonged recovery time, and are not reversible once the target weight of the patient is reached.

Another procedure used is vertical stapled gastroplasty. This procedure involves incision of the anterior abdominal wall and creation of a 10-15 ml pouch from the proximal stomach by use of 3-4 staples. This procedure also has numerous complications including rupture of the staple line, infection of the surgical incision, post operative hernias and the like. Moreover, due to the large amount of fat tissue in the anterior abdominal wall in the typical patient on whom this procedure is performed, poor healing of the operative wound may result. Furthermore prolonged post-operative bed rest after such extensive surgery predisposes obese patients to the development of deep vein thrombosis and possible pulmonary emboli, some with a potentially lethal outcome.

Thus, there is a need for a device, method, and system to reduce weight that is less traumatic, has less recovery time, is reversible, not complicated, and is simple to perform. Additionally, there is a need for a device, method, and system that is available to less obese people that would like to lose only 20 to 50 pounds.

### BRIEF DESCRIPTION OF THE INVENTION

A solution is provided for modifying the location at which bodily fluids interact with nutrients in a gastrointestinal tract having a conduit with a first end and a second end. The first end is configured to divert bodily fluids from an entrance within a gastrointestinal tract to a location downstream from the entrance. The solution also provides for a means for attaching the second end to the entrance.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

In the drawings:

FIG. 1 is an illustration of the digestive system.

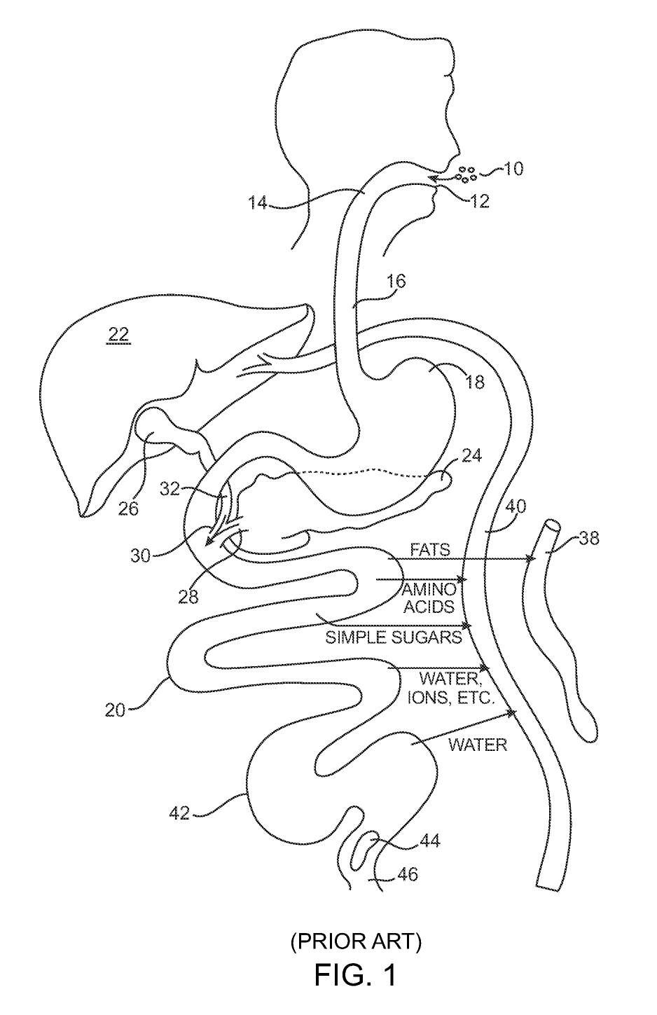

FIG. 2A is a diagram illustrating an embodiment of the present invention.

FIGS. 2B and 2C are diagrams of an embodiment of the cap 204.

FIG. 3 is a graph illustrating data obtained from testing of the device in a pig.

FIG. 4 illustrates the device in accordance with an alternative embodiment of the present invention.

FIG. 5 is a block diagram illustrating a method of using the device in accordance with an embodiment of the invention.

### DETAILED DESCRIPTION

Embodiments of the present invention are described herein in the context of a minimally invasive gastrointestinal bypass. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one, implementation to another and from one developer to another. Moreover, it will be appreciated that such a development effort might be complex and time-consuming, but would nevertheless be a routine undertaking of engineering for those of ordinary skill in the art having the benefit of this disclosure.

The present invention is a system, method, device, and apparatus to treat obesity through gastrointestinal bypass. By bypassing bodily fluids such as enzymatic, food, and other fluids to a location distal the GI tract, less food will be absorbed by the body and more food will be excreted, which results in weight loss.

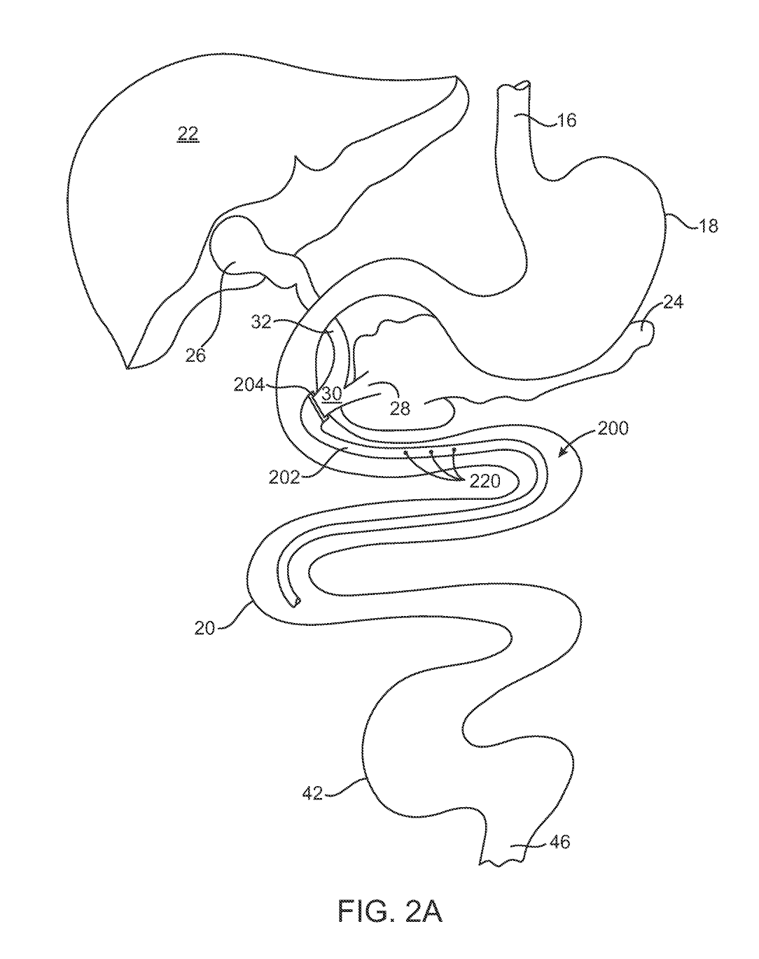

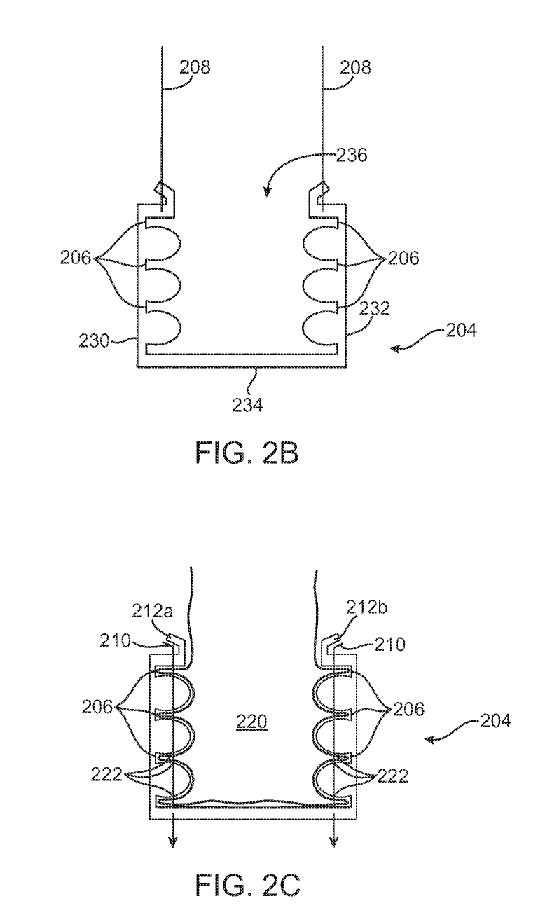

FIG. 2A is a diagram illustrating an embodiment of the present invention. The device, generally numbered as 200, shortens the effective absorption length of the bowel or GI tract. The effective absorption is the amount of digested food that is absorbed by the body. By bypassing the bodily fluids in the GI tract, such as bile and pancreatic enzymes, to a location further downstream within the GI tract, nutrients from the food fluid will not be absorbed by the enzymes or emulsifying reagents in the body as it travels from the stomach and through the intestine. This will also reduce the absorption time of the food fluids into the body. Thus, the effective absorption of nutrients from the food fluids is decreased whereby most of the food fluids are excreted which results in the patient's weight loss.

The device 200 may have a cap 204 and a conduit 202 that are delivered through the GI tract and removably attached to the small intestine 20. The conduit 202 may be a flexible tube having a first end 252 configured to divert enzymatic fluids to a location significantly further down the GI tract. The conduit 202 may be large enough in diameter such that the enzymes may pass through the flexible tube without forming stones or becoming infected. In an alternative embodiment, the conduit may contain a plurality of apertures 220 to allow some enzymatic fluids to pass through to prevent injury or death to the patient should the conduit become clogged. The conduit 202 may also have a side port (not shown) to allow fluids, such as saline, or gas to pass through the conduit to extend, straighten, or unfurl the conduit into the GI tract as will be further described below. This may also ensure that the lumen of the conduit is free and clear of any obstructions. However, the conduit may unfurl itself by having the bile and pancreatic secretions fill the conduit or through intestinal peristalsis.

The length of the conduit 202 at the first end 252 is adjustable depending on the amount of weight the patient would like to lose. Since the amount of malabsorption resulting from placement of the conduit 202 is related to the length of the bowel pass by the conduit, adjustments in the length of the conduit 202 would be beneficial. Thus, the location of where the enzymatic fluids are to exit in the GI tract may be variable and may be determined by the doctor. The conduit 202 may be shortened by trimming its length prior to insertion into a patient's body. Additionally, a filamentous member may be attached to the conduit such that when the filamentous member is pulled, the conduit 202 will shorten in an accordion style. The ability to adjust the length of the conduit 202 allows for the adjustment of the weight loss effects from the device as the patient reaches its target or desired weight.

FIGS. 2B and 2C are diagrams of an embodiment of the cap 204. In an embodiment of the present invention, the device does not necessarily need the cap 204. Rather, the a second end 250 of the conduit 202, may be attached to the Ampula of Vater 30 through sutures, staples, or hooks. The cap has a first side 230, a second side 232, and a bottom 234 thereby forming a cavity 236 to receive a portion of the GI tract as further described in detail below.

The cap 204 may attach the conduit 202 to the Ampula of Vater 30. The cap 204 may be made of a transparent material so that a user may see through it to accurately position the cap. If a patient has two ducts, then the cap 204 may be formed to cover the ducts. In an alternative embodiment, the conduit 202 may comprise two separate caps to cover both ducts. The cap 204 may have a plurality of channels 206 to capture the tissue 220 around the Ampula of Vater 30 and at least one wire 208 to secure the tissue in the cap 204. In use, the cap 204 is positioned around the Ampula of Vater 30 and vacuum suction is used to suction the tissue 220 into the cap 204. The wires 208 may then be pushed downward through wire holes 222 in the channels 206 to secure the tissue in place. The wires 208 may be bent at a first end 210 and held in place by hooks 212a, 212b.

The tissue 220 may be placed into the cap through other means, such as the use of a corkscrew or a multiple-tined piercing device. When using multiple-tined piercing device, the tines are kept together while inserted into the patient to prevent damage to the patient. The tines are expanded and contracted to grab the tissue around the Ampula of Vater. The tissue is then pulled into the cap, the tines are expanded to release the tissue, and the multiple-tined piercing device is again contracted to retract it out of the patient's body.

The wires 208 may be held in position by any other means, such as the channels may have barbs to retain the wire, the wire may have a barb to retain it against the tissue, the hook may be twistable to secure the wire in place, and the like. Although the wire 208 is illustrated as two separate wires, the wire 208 may be a single piece of wire within the cap 204.

The device 200 may be attached to the Ampula of Vater by other means such as staples, sutures, or hooks. The device 200 may be made of any material that may be absorbable by the body such as polyglycolated resins, polygalactic acid materials, and other similar materials or non-absorbable materials such as silicone, polyethylene, polypropylene, butylated rubber, latex, and the like. If the device 200 is made of non-absorbable material, the device 200 may be easily removed from the patient when a target or ideal weight is obtained. The device may also be easily removed with an endoscope through the patient's mouth. Alternatively, the cap, or other means of attachment used to secure the device, may be made of an absorbable material to allow the remaining device to pass through the anal canal. In another embodiment, the conduit may be made of a semi-permeable material, such as Goretex, to selectively allow certain bodily fluids to pass through the conduit. For instance, the semi-permeable material may allow water to enter the conduit to assist in the flow of fluids through the conduit.

By modifying the location at which enzymatic fluids interact with nutrients from food fluids in the GI tract, less nutrients from the food fluids will be absorbed by the body, the effectiveness of enzyme and emulsifying reagent reacting with the food fluids will be decreased, and more of the food fluids will be excreted resulting in a weight loss. Thus, the proportion of absorbed food fluids to excreted food fluids is changed which results in the weight loss. Additionally, as further discussed below, the patient may continue to consume the same amount of food, and use of the device will allow for a weight loss as well as the maintenance of the weight.

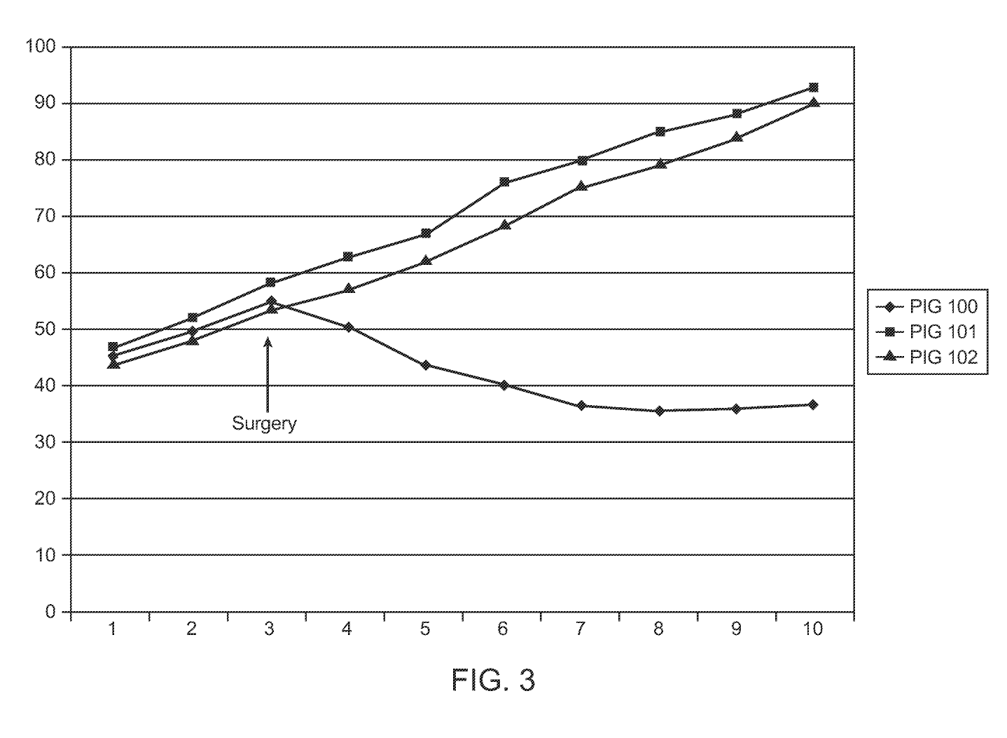

FIG. 3 is a graph illustrating data obtained from testing the device in a pig. The Y-axis is weight in Kilograms and the X-axis is time in weeks. Pigs 100, 101, and 102 were allowed to consume the same amount of food throughout the testing period. Pigs 101 and 102 were controls and did not contain the device. Rather, the device was inserted into Pig 100 at week 3 at which time all the pigs weighed between 54-59 kilograms. After the surgery, Pig 100 rapidly lost weight in weeks 3 through 7 going from 55 kilograms to 36 kilograms while pigs 101 and 102 continued to gain weight. Data after week 7 indicates that Pig 100 was able to continually maintain a constant weight at about 35 kilograms for several weeks thereafter. Although Pig 100 continued to consume the same amount of food each day similar to Pigs 101 and 102, Pig 100 still lost weight and was able to maintain the weight.

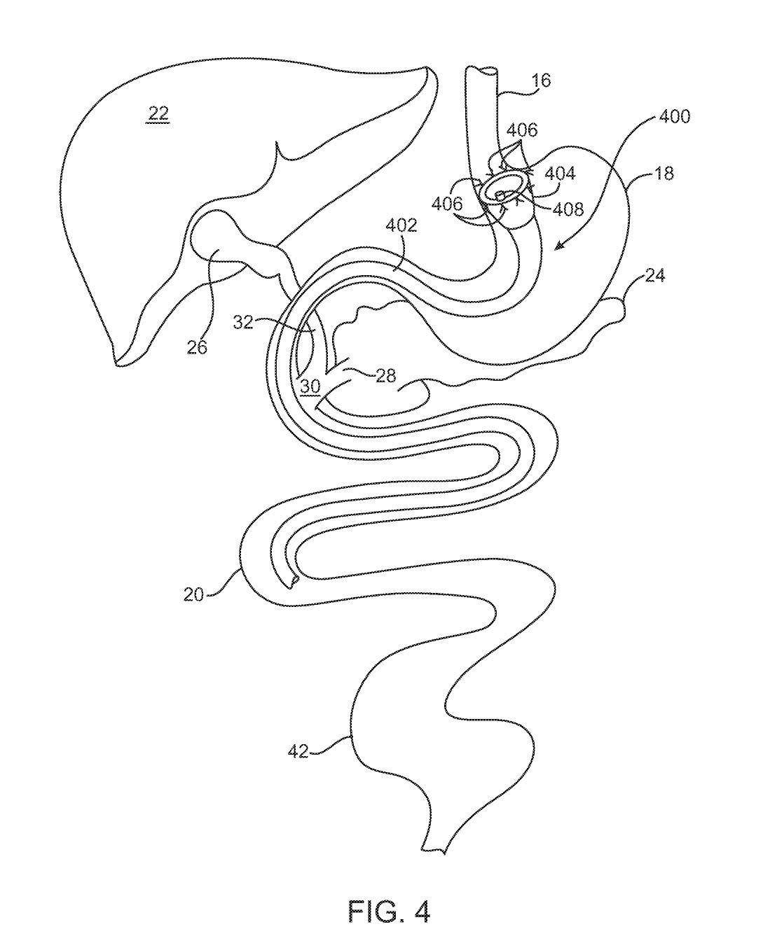

FIG. 4 illustrates the device in accordance with an alternative embodiment of the present invention. The device, generally numbered as 400, has a conduit 402 and a cap 404. The device 400 may be positioned within the stomach to capture food fluids and deposit the food fluids to a location distal the GI tract. Thus, the body will absorb less food and more food will be excreted, which results in weight loss.

The conduit 402 is similar to the conduit described above with reference to FIG. 2A and will not be discussed further. The cap 404 may be an expandable funnel shaped cap having a plurality of retention wires 406. The retention wires 406 aid in securing the cap 404 in its position by grasping onto the wall of the bowel or GI tract. Although the embodiment is described and shown with the use of retention wires, other means of attachment may be used such as sutures, staples, or hooks. The implantation site of the device 400 determines the volume of stomach 18 the patient will have or need to achieve the target or desired weight. Thus, the size of the cap 404 may be varied in diameter based upon each patient's requirements.

In an alternative embodiment, the cap 404 may be asymmetrically shaped such that the stomach anterior, or fundus, is included in the cap 404. Thus, when the funnel is filled, a sensation of fullness is perceived and causes satiety. The cap may also be shaped to fill the antrum in the stomach to also provide a sense of fullness and allow hormonal feedback of satiety.

The cap 404 may also have a side port 408 to allow fluids, such as saline, or gas to expand or contract the cap 404. Thus, the cap 404 may be easily adjusted to decrease or increase the volume of the stomach 18.

The cap 404 may also have a grid or mesh positioned on top of or within the cap 404 to prevent large materials from clogging or plugging up the conduit. The large materials may either pass through the GI tract or be expelled by the patient by vomiting.

The device 400 may be made of any absorbable material such as polyglycolated resins, polygalactic acid materials, and other similar materials or non-absorbable materials such as silicone, polyethylene, polypropylene, butylated rubber, latex, and the like. If the device 400 is made of non-absorbable material, it may be easily removed from the patient when a target or ideal weight is obtained. The device may also be easily removed with an endoscope through the patient's mouth. Alternatively, the cap, or other means of attachment used to secure the device, may be made of an absorbable material to allow the remaining device to pass through the anal canal. In another embodiment, the conduit may be made of a semi-permeable material, such as Goretex, to selectively allow certain bodily fluids to pass through the conduit. For instance, the semi-permeable material may allow water to enter the conduit to assist in the flow of fluids through the conduit.

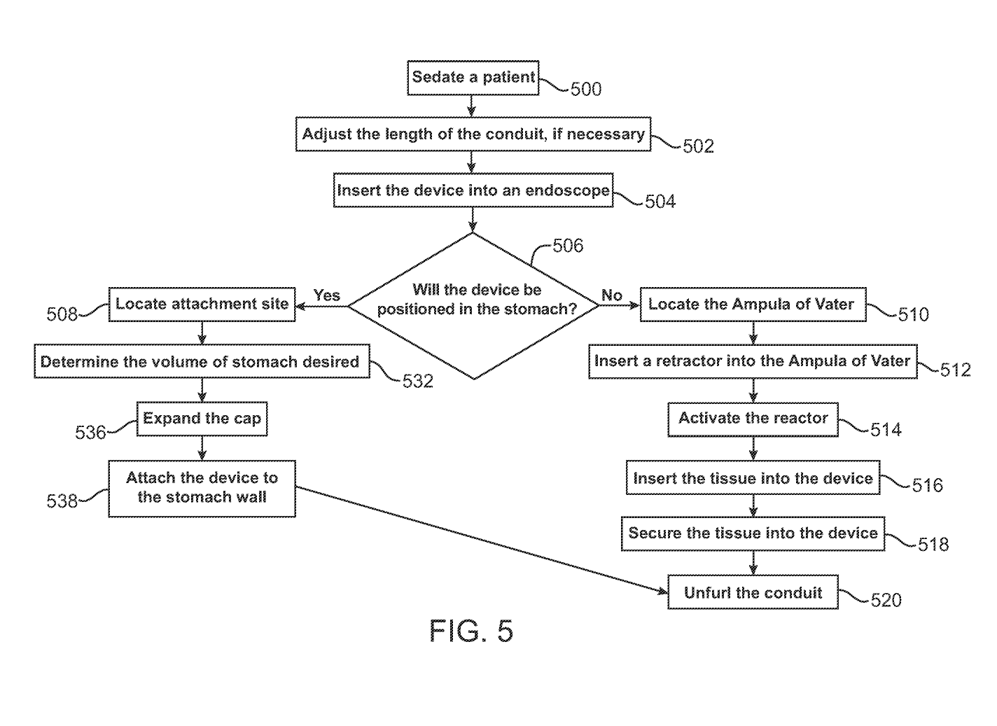

FIG. 5 is a block diagram illustrating a method of using the device in accordance with an embodiment of the invention. The device may be inserted into a patient without major surgery, incisions, or the use of general anesthesia. Rather, the patient may be sedated at 500 when the device is to be delivered through the mouth of a patient. The length of the device may be adjusted at 502, if necessary, based upon the amount of weight the patient would like to lose. The length may be trimmed or cut by any means such as with scissors. The device is then inserted into an endoscope at 504. The device may be inserted either prior to inserting the endoscope into the patient's mouth or after insertion of the endoscope into the patient's mouth. The insertion and use of an endoscope is well known and will not be described herein so as not to overcomplicate the present disclosure. However, the device may be formed in any shape possible that would allow for the easiest and safest means to place the device into the patient. By way of example only, and not intended to be limiting, the device may be rolled-up onto itself, the device may be folded into a fan shape, or the device may be folded into a zigzag shape before insertion into the patient's body.

If the device is to be positioned in the stomach at 506, the location of attachment to the wall of the bowel is located at 508 and the desired volume of stomach is determined at 532. The cap may be expanded at 536 and attached to the wall of the stomach at 538. The cap may be attached with retention wires, but other means of attachment may be used such as sutures, staples, or hooks. The cap may alai have a side port to allow fluids, such as saline, or gas to expand or contract the cap. Thus, the cap may be easily adjusted to decrease or increase the volume of the stomach. The conduit is unfurled at 520. The conduit may also have a side port to allow fluids, such as saline, or gas to pass through the conduit to extend, straighten, or unfurl the conduit into the GI tract. This ensures that the lumen of the conduit is free and clear of any obstructions. However, the conduit may unfurl itself by having the bile and pancreatic secretions fill the conduit or through intestinal peristalsis.

If the device is not positioned within the stomach at 506, the Ampula of Vater is located at 510 using the endoscope. A retractor is inserted into the Ampula of Vater at 512. The retractor may have an expandable balloon or a fenestrated tube that may be activated with a vacuum suction to suction the tissue around the Ampula into the cap. However, other methods of retraction are possible such as a corkscrew that may be screwed into the tissue or a multiple-tined piercing device. When using multiple-tined piercing devices, the tines are kept together while inserted into the patient to prevent damage to the patient. The tines are expanded and contracted to grab the tissue around the Ampula of Vater. The tissue is then pulled into the device, the tines are expanded to release the tissue, and the multiple-tined piercing device is again contracted to retract it out of the patient's body.

The retractor is activated at 514 to insert the tissue into the device at 516. If a vacuum suction is used, the vacuum is applied to suction and retain the tissue into the cap. The tissue is then secured in the device at 518. If a cap is used with the device, the tissue will be inserted into the cap and secured with wires that are pushed downward through wire holes in the channels to secure the tissue in place. The wires may be bent at a first end and held in place by hooks on the cap.

The wires may be held in position by any other means, such as the channels may have a barb to retain the wire, the wire may have a barb to retain it again the tissue, the hook may be twistable to secure the wire in place, and the like. However, the device may also be attached to the Ampula of Vater by other means such as staples, sutures, or hooks.

The conduit is unfurled at 520. The conduit may have a side port to allow fluids, such as saline, or gas to pass through the conduit to extend, straighten, or unfurl the conduit into the GI tract. This ensures that the lumen of the conduit is free and clear of any obstructions. However, the conduit may unfurl itself by having the bile and pancreatic secretions fill the conduit or through intestinal peristalsis.

If the patient's target or ideal weight has been reached and the patient would like to remove the device, the device may be easily removed from the patient's body. Alternatively, the device may remain in the patient's body, but the length of the conduit may be adjusted.

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

1. A system for modifying the location at which bodily fluids interact with nutrients in a gastrointestinal tract, comprising:
a conduit having a first end and a second end, said first end configured to divert bodily fluids from an entrance within a gastrointestinal tract to a location downstream from said entrance; and
means for attaching said second end to said entrance.

2. The system of claim 1 wherein said conduit comprises a flexible tube having a tube length.

3. The system of claim 2 wherein said tube length is adjustable.

4. The system of claim 1 wherein said conduit is delivered into a patient through the gastrointestinal tract.

5. The system of claim 1 wherein said conduit and said means for attaching are made of an absorbable material.

6. The system of claim 1 wherein said conduit further comprises a plurality of apertures.

7. The system of claim 1 wherein said entrance is the Ampula of Vater.

8. The system of claim 7 wherein said bodily fluids comprise a bile secretion.

9. The system of claim 7 wherein said bodily fluids comprise a pancreatic secretion.

10. The system of claim 7 wherein said entrance further comprises at least one duct.

11. The system of claim 1 wherein said means for attaching comprises a cap.

12. The system of claim 11 wherein said cap is removably affixed to said entrance.

13. The system of claim 11 wherein said cap is permanently affixed to said entrance.

14-22. (canceled)

23. The system of claim 1 wherein the diversion of said bodily fluids to said downstream location operates to reduce an amount of bodily fluids that interact with the nutrients.

24. The system of claim 1 wherein the diversion of said bodily fluids to said downstream location operates to alter an amount of nutrients absorbed by the gastrointestinal tract.

25. The system of claim 1 wherein the diversion of said bodily fluids to said downstream location operates to control and stabilize a patient's weight.

26. The system of claim 1 wherein said first end is positioned such that an amount of interaction between the bodily fluids and said nutrients is reduced.

27. The system of claim 26 wherein an absorption time between the bodily fluids and the nutrients is reduced.

28. A device for shortening an effective absorption length of a bowel, comprising:
a conduit having a first end configured to divert a bodily fluid to a location in a gastrointestinal tract distally from an entrance; and
a cap coupled to a conduit second end to attach said conduit to said entrance.

29. The device of claim 28 wherein said conduit comprises a flexible tube having a tube length.

30. The device of claim 29 wherein said tube length is adjustable.

31. The device of claim 28 wherein said bodily fluid further comprises a bile secretion

32. The device of claim 28 wherein said bodily fluid further comprises a pancreatic secretion.

33. The device of claim 28 wherein said entrance comprises at least one duct.

34. The device of claim 28 wherein said entrance is the Ampula of Vater.

35. The device of claim 28 wherein said cap is removable from said entrance.

36. The device of claim 28 wherein said cap is permanently attached to said entrance.

37. The device of claim 28 wherein said conduit is delivered into a body through the gastrointestinal tract.

38-44. (canceled)

45. A method of shortening an effective absorption length of a bowel, comprising:
inserting a conduit into a patient's mouth, the conduit having a cap at a first end of said conduit;
locating a fluid entrance in the digestive tract of the patient;
positioning a cap over the fluid entrance; and
affixing said cap over said entrance,
wherein a second end of said conduit diverts, by a predetermined distance, fluid entering said fluid entrance to a location in the digestive tract that is downstream from said fluid entrance.

46. The method of claim 45 wherein said inserting further comprises attaching a conduit onto an endoscope.

47. The method of claim 45 wherein said inserting further comprises adjusting a length of said conduit.

48. The method of claim 45 wherein said conduit comprises a flexible tube.

49. The method of claim 45 wherein said entrance comprises at least one duct.

50. The method of claim 45 wherein said entrance is the Ampula of Vater.

51. The method of claim 45 further comprising removing said cap when an ideal weight is achieved.

52. The method of claim 45 wherein said conduit and said cap are made of an absorbable material.

53. The method of claim 45 wherein said cap is made of a transparent material.

54. The method of claim 45 wherein said conduit further comprises a plurality of apertures.

55. The method of claim 45 wherein said affixing further comprises suctioning said cap to said entrance.

56. The method of claim 55 further comprising securing said cap to said entrance with a wire.

57. The method of claim 45 wherein said affixing further comprises securing said cap to said entrance with at least one staple.

58. The method of claim 45 wherein said affixing further comprises screwing said cap to said entrance.

59. The method of claim 45 wherein said locating further comprises extending said conduit.

60. The method of claim 59 wherein said extending further comprises inserting a saline solution through said cap.

61. The method of claim 59 wherein said extending further comprises inserting air through said cap.

62. The method of claim 45 wherein said affixing further comprises extending said conduit.

63. The method of claim 60 wherein said extending further comprises inserting a saline solution through said cap.

64. The method of claim 60 wherein said extending further comprises inserting air through said cap.

65-117. (canceled)
